(19) United States
(12) Patent Application Publication
(10) Pub. No.: US 2026/0102321 A1
(43) Pub. Date: Apr. 16, 2026

(54) METHOD FOR PRODUCING SOLID POWDER COSMETIC AND SOLID POWDER COSMETIC

(71) Applicant: TOKIWA CORPORATION, Gifu (JP)

(72) Inventor: Yuta KODAMA, Saitama (JP)

(73) Assignee: TOKIWA CORPORATION, Gifu (JP)

(21) Appl. No.: 19/344,967

(22) Filed: Sep. 30, 2025

(30) Foreign Application Priority Data
Oct. 10, 2024 (JP) 2024-178287

(51) Int. Cl. A61K 8/02; A61Q 1/12

(52) U.S. Cl. CPC A61K 8/022; A61Q 1/12; A61K 2800/48; A61K 2800/621; A61K 2800/651

(57) ABSTRACT

The method for producing a solid powder cosmetic includes: a step of preparing a slurry containing a cosmetic base material including a powder component and an oily component, and a containing water; and a step of filling the slurry into a container to mold the slurry by removing the dispersion medium, in which the powder component includes (A) at least one plate-like powder selected from the group consisting of (A1) a plate-like powder composed of alumina single crystal and (A2) a plate-like powder in which a surface of alumina is coated with a metal oxide, and (B) a spherical powder, the cosmetic base material further includes (C) a water-soluble thickener, and the oily component includes (D) a water-holding oil agent.

### TECHNICAL FIELD

The present disclosure relates to a method for producing a solid powder cosmetic and to a solid powder cosmetic.

### BACKGROUND

A solid powder cosmetic composed of powder components and oily components is used as a makeup cosmetic such as blush, eye shadow, foundation, and face powder. Many of these cosmetics are used in a form stored in compacts container from the viewpoint of portability. Such solid powder cosmetics are produced by a dry production method in which a cosmetic base material obtained by mixing a powder component and an oily component is filled into a container such as a metal pan and compression-molded, or by a wet production method in which a volatile organic solvent such as a volatile hydrocarbon oil or a volatile lower alcohol is added as a dispersion medium to the cosmetic base material to form a slurry, the slurry is filled into a container, and then the dispersion medium is removed and compression-molded.

A solid powder cosmetic obtained by the wet production method is likely to provide a favorable feeling of use in terms of spreadability at the time of application. In recent years, with environmental considerations, a wet production method using water as a solvent has also been studied (see, for example, Japanese Unexamined Patent Publication No. 2010-37207).

### SUMMARY

However, in comparison with the case of using a volatile organic solvent, a wet production method using water makes slurry filling difficult, tends to increase the amount of dispersion medium, and causes cracks on the surface, voids inside, and gaps between the outer periphery and the container in the cosmetic after drying. These defects lead to problems in usability such as a tendency for powder scattering when the solid powder cosmetic is taken up with an applicator and difficulty in taking an appropriate amount, and also contribute to a reduction in impact resistance of the solid powder cosmetic.

In the method for producing a solid powder cosmetic described in Japanese Unexamined Patent Publication No. 2010-37207 above, improvement in impact resistance of the solid powder cosmetic is achieved by using a slurry containing a specific powder component, a specific oily component, a polyhydric alcohol, and a specific surfactant in specific proportions. However, according to studies by the inventors, it has been found that a solid powder cosmetic containing a polyhydric alcohol may undergo a significant decrease in hardness in a high-humidity environment. Since an excessive decrease in hardness leads to a decrease in drop strength, changes in feeling of use, or the like, even in the above-described related art, there is still room for improvement in terms of storage stability of a solid powder cosmetic.

On the other hand, there are also demands related to a makeup finish feeling for a solid powder cosmetic. Specifically, for example, a face powder used for finishing makeup is required to have good spreadability when applied on the skin, as well as good adhesion to the skin, including reduced dryness or powder scattering and ease of blending, and a transparent finish. It is known that improvement in spreadability on the skin can be achieved by increasing the blending amount of spherical powder such as silica or nylon; however, in this case, adhesion to the skin tends to decrease, and furthermore, problems of powder scattering when taken up with an applicator and the above-described decrease in impact resistance are also likely to occur. Since face powder or the like is mainly used in a manner of being taken up with an applicator such as a brush and applied to the skin, excellent usability in which powder scattering is reduced and an appropriate amount can be easily taken is required.

The present invention has been made in view of the above circumstances, and an object of the present invention is to provide a solid powder cosmetic having excellent usability (reduced powder scattering and ease of taking an appropriate amount), an excellent feeling of finish (adhesion to the skin and a transparent finish), sufficient impact resistance, and sufficient storage stability, and to provide a method for producing a solid powder cosmetic by which such a solid powder cosmetic can be obtained by a wet production method using water.

The present invention provides the following methods [1] to [8] for producing a solid powder cosmetic.

[1] A method for producing a solid powder cosmetic including: a step of preparing a slurry containing a cosmetic base material including a powder component and an oily component, and a dispersion medium containing water; and a step of filling the slurry into a container to mold the slurry by removing the dispersion medium, in which the powder component includes (A) at least one plate-like powder selected from the group consisting of (A1) a plate-like powder composed of alumina single crystal and (A2) a plate-like powder in which a surface of alumina is coated with a metal oxide, and (B) a spherical powder, the cosmetic base material further includes (C) a water-soluble thickener, and the oily component includes (D) a water-holding oil agent.

[2] The method for producing a solid powder cosmetic according to [1], in which the content of polyhydric alcohol in the cosmetic base material is less than 0.1% by mass based on the cosmetic base material. [3] The method for producing a solid powder cosmetic according to [1] or [2], in which the content of the component (D) in the cosmetic base material is 50% by mass or less based on the total amount of the oily component.

[4] The method for producing a solid powder cosmetic according to any one of [1] to [3], wherein the component (C) includes at least one thickener selected from the group consisting of acrylic acid copolymer and a clay mineral.

[5] The method for producing a solid powder cosmetic according to any one of [1] to [3], wherein the component (C) includes at least one acrylic acid copolymer selected from the group consisting of (hydroxyethyl acrylate/sodium acryloyldimethyltaurate) copolymer, (sodium acrylate/sodium acryloyldimethyltaurine) copolymer, and sodium acrylate graft starch.

[6] The method for producing a solid powder cosmetic according to any one of [1] to [5], wherein the component (B) includes spherical particles surface-treated with a higher alcohol.

[7] The method for producing a solid powder cosmetic according to any one of [1] to [6], wherein the content of semi-solid oil in the cosmetic base material based on the total amount of the cosmetic base material is 1 to 6% by mass.

[8] The method for producing a solid powder cosmetic according to [2],
wherein the content of the component (D) in the cosmetic base material is 50% by mass or less based on the total amount of the oily component,
wherein the component (C) includes at least one acrylic acid copolymer selected from the group consisting of (hydroxyethyl acrylate/sodium acryloyldimethyltaurate) copolymer, (sodium acrylate/sodium acryloyldimethyltaurine) copolymer, and sodium acrylate graft starch,
wherein the component (B) includes spherical particles surface-treated with a higher alcohol,
wherein the content of semi-solid oil in the cosmetic base material based on the total amount of the cosmetic base material is 1 to 6% by mass.

The present invention also provides the following solid powder cosmetics [9] to [16].

[9] A solid powder cosmetic including: a powder component; and an oily component, in which the powder component includes (A) at least one plate-like powder selected from the group consisting of (A1) a plate-like powder composed of alumina single crystal and (A2) a plate-like powder in which a surface of alumina is coated with a metal oxide, and (B) a spherical powder, the solid powder cosmetic further includes (C) a water-soluble thickener, and the oily component includes (D) a water-holding oil agent.

[10] The solid powder cosmetic according to [9], in which the content of polyhydric alcohol in the solid powder cosmetic is less than 0.1% by mass based on the solid powder cosmetic.

[11] The solid powder cosmetic according to [9] or [10], in which the content of the component (D) in the solid powder cosmetic is 50% by mass or less based on the total amount of the oily component.

[12] The solid powder cosmetic according to any one of [9] to [11], wherein the component (C) includes at least one thickener selected from the group consisting of acrylic acid copolymer and a clay mineral.

[13] The solid powder cosmetic according to any one of [9] to [11], wherein the component (C) includes at least one acrylic acid copolymer selected from the group consisting of (hydroxyethyl acrylate/sodium acryloyldimethyltaurate) copolymer, (sodium acrylate/sodium acryloyldimethyltaurine) copolymer, and sodium acrylate graft starch.

[14] The solid powder cosmetic according to any one of [9] to [13], wherein the component (B) includes spherical particles surface-treated with a higher alcohol.

[15] The method for producing a solid powder cosmetic according to any one of [9] to [14], wherein the content of semi-solid oil in the solid powder cosmetic based on the total amount of the solid powder cosmetic is 1 to 6% by mass.

[16] The method for producing a solid powder cosmetic according to [10],
wherein the content of the component (D) in the solid powder cosmetic is 50% by mass or less based on the total amount of the oily component,
wherein the component (C) includes at least one acrylic acid copolymer selected from the group consisting of (hydroxyethyl acrylate/sodium acryloyldimethyltaurate) copolymer, (sodium acrylate/sodium acryloyldimethyltaurine) copolymer, and sodium acrylate graft starch,
wherein the component (B) includes spherical particles surface-treated with a higher alcohol,
wherein the content of semi-solid oil in the solid powder cosmetic based on the total amount of the solid powder cosmetic is 1 to 6% by mass.

According to the present invention, it is possible to provide a solid powder cosmetic having excellent usability (reduced powder scattering and ease of taking an appropriate amount), an excellent feeling of finish (adhesion to the skin and a transparent finish), sufficient impact resistance, and sufficient storage stability, and to provide a method for producing a solid powder cosmetic by which such a solid powder cosmetic can be obtained by a wet production method using water.

### DETAILED DESCRIPTION

### [Method for Producing Solid Powder Cosmetic]

The method for producing a solid powder cosmetic of the present embodiment includes: Step A of preparing a slurry containing a cosmetic base material including a powder component and an oily component, and a dispersion medium containing water, and Step B of filling the slurry into a container to mold the slurry by removing the dispersion medium.

In the above cosmetic base material, the powder component includes (A) at least one plate-like powder (sometimes referred to as a component (A)) selected from the group consisting of (A1) a plate-like powder composed of alumina single crystal (sometimes referred to as a component (A1)) and (A2) a plate-like powder (sometimes referred to as a component (A2)) in which a surface of alumina is coated with a metal oxide, and (B) a spherical powder (sometimes referred to as a component (B)), and further includes (C) a water-soluble thickener (sometimes referred to as a component (C)), and the oily component includes (D) a water-holding oil agent (sometimes referred to as a component (D)).

According to the method for producing a solid powder cosmetic of the present embodiment, by having the above configuration, it is possible to obtain a solid powder cosmetic having excellent usability (reduced powder scattering and ease of taking an appropriate amount), an excellent feeling of finish (adhesion to the skin and a transparent finish), sufficient impact resistance, and sufficient storage stability.

### (Step A)

First, the components constituting the cosmetic base material contained in the slurry will be described.

### <Powder Component>

In the present embodiment, the powder component includes a component (A) and a component (B).

The component (A) can be used without particular limitation as long as it is used in cosmetics.

The component (A1) may be, for example, a commercially available product such as “RonaFlair (registered trademark) White Sapphire” (manufactured by Merck KGaA, product name).

Examples of the component (A2) include an alumina-based pearlescent agent in which a surface of plate-like alumina is coated with a metal oxide such as titanium oxide, tin oxide, or iron oxide. The component (A2) may be, for example, a commercially available product such as “Ronastar (registered trademark) Golden Lights” (manufactured by Merck KGaA, product name) or “Mirinae series” (manufactured by CQV Co., Ltd., product name).

An average particle diameter of the component (A) may be, from the viewpoints of adhesion to the skin, a transparent finish, and suppression of powder scattering, 2 to 100 μm, 4 to 80 μm, or 5 to 45 μm. An average particle diameter of the component (A1) may be 2 to 50 μm, 4 to 40 μm, or 5 to 30 μm. An average particle diameter of the component (A2) may be 5 to 100 μm, 8 to 80 μm, or 15 to 45 μm. In the present specification, the “average particle diameter” refers to a D50 value based on a volume distribution measured using a laser diffraction particle size distribution analyzer.

The component (A) can be used alone or in combination of two or more thereof.

The content of the component (A) in the cosmetic base material may be, from the viewpoints of improving adhesion to the skin and transparency of finish, based on the total amount of the cosmetic base material, 0.1 to 75% by mass, 5 to 60% by mass, or 15 to 45% by mass.

The cosmetic base material may include the component (A1) as the component (A) from the viewpoints of improving adhesion to the skin and transparency of finish.

When the cosmetic base material includes the component (A1), the content of the component (A1) in the cosmetic base material may be, from the viewpoints of improving transparency and glossiness of finish, based on the total amount of the component (A), 0.1% by mass or more, 10% by mass or more, 25% by mass or more, 40% by mass or more, 75% by mass or more, 80% by mass or more, or 100% by mass.

When the cosmetic base material includes the component (A2), the content of the component (A2) in the cosmetic base material may be, from the viewpoints of improving transparency and pearly feeling of finish, based on the total amount of the component (A), 15% by mass or more, 25% by mass or more, 55% by mass or more, or 75% by mass or more.

The component (B) can be used without particular limitation as long as it is used in cosmetics.

Examples of the component (B) include: inorganic powders such as silica, calcium carbonate, magnesium carbonate, and barium sulfate; and organic powders such as nylon powder, polymethyl methacrylate powder, acrylonitrile-methacrylic acid copolymer powder, vinylidene chloride-methacrylic acid copolymer, polyethylene powder, polystyrene powder, organopolysiloxane elastomer powder, polymethylsilsesquioxane powder, urethane powder, wool powder, silk powder, cellulose powder, amylose powder, N-acyl lysine powder, and starch-derived powder.

The component (B) may be surface-treated. The surface treatment can be employed without particular limitation as long as it is used in cosmetics, and examples thereof include surface treatment with a metal soap, a higher alcohol, a higher fatty acid, an oil or fat, a wax, a silicone compound, a fluorine compound, a surfactant, or a dextrin fatty acid ester.

The cosmetic base material may include spherical particles, as the component (B), surface-treated with a higher alcohol, from the viewpoints of suppression of powder scattering and improvement in adhesion to the skin.

The higher alcohol may be an alcohol having 12 to 18 carbon atoms, and may be a saturated aliphatic alcohol having 12 to 18 carbon atoms. Examples of the higher alcohol include cetanol and stearyl alcohol.

An average particle diameter of the component (B) may be, from the viewpoints of good spreadability on the skin and ease of taking an appropriate amount, 2 to 50 μm, 4 to 30 μm, or 5 to 15 μm.

The component (B) can be used alone or in combination of two or more thereof.

The content of the component (B) in the cosmetic base material may be, from the viewpoints of good spreadability on the skin and ease of taking an appropriate amount, based on the total amount of the cosmetic base material, 1% by mass or more, 3% by mass or more, 5% by mass or more, 10% by mass or more, 12% by mass or more, or 15% by mass or more, and may be, from the viewpoints of suppression of powder scattering and impact resistance, based on the total amount of the cosmetic base material, 40% by mass or less, 30% by mass or less, 25% by mass or less, or 20% by mass or less.

From the viewpoints of suppression of powder scattering and improvement in adhesion to the skin (particularly ease of blending with the skin) and transparency of finish in a well-balanced manner, the total content of the component (A) and the component (B) in the cosmetic base material may be, based on the total amount of the cosmetic base material, 15 to 75% by mass, 25 to 55% by mass, or 30 to 50% by mass.

From the viewpoints of suppression of powder scattering and improvement in adhesion to the skin (particularly ease of blending with the skin) and transparency of finish in a well-balanced manner, a ratio [(A)/(B)] of the content of the component (A) to the content of the component (B) in the cosmetic base material may be 0.1 to 20, 0.3 to 15, or 0.5 to 10.

From the viewpoints of ease of adjusting an amount adhering to the applicator when taken up with the applicator, ease of taking an appropriate amount, transparency of finish, and good spreadability, the cosmetic base material may include inorganic powders as the component (B). The inorganic powders may include spherical silica.

From the viewpoints of ease of taking an appropriate amount and improvement in adhesion to the skin and good spreadability in a well-balanced manner, the content of spherical silica in the cosmetic base material may be, based on the total amount of the cosmetic base material, 0.01 to 50% by mass, 1 to 30% by mass, 3 to 25% by mass, or 4 to 20% by mass.

From the viewpoints of suppression of powder scattering and improvement in ease of taking an appropriate amount, transparency of finish, good spreadability, and moldability in a well-balanced manner, the total content of the component (A) and spherical silica in the cosmetic base material may be, based on the total amount of the cosmetic base material, 15 to 75% by mass, 25 to 55% by mass, or 30 to 50% by mass.

From the viewpoints of suppression of powder scattering and improvement in adhesion to the skin, the component (B) may include spherical silica surface-treated with a higher alcohol (higher alcohol-treated spherical silica).

From the viewpoints of suppression of powder scattering and improvement in ease of taking an appropriate amount, adhesion to the skin, transparency of finish, good spreadability, and moldability in a well-balanced manner, the content of higher alcohol-treated spherical silica in the cosmetic base material may be, based on the total amount of the cosmetic base material, 2 to 30% by mass, 3 to 20% by mass, or 4 to 16% by mass.

From the viewpoints of good spreadability, ease of taking an appropriate amount, and moldability, the component (B) may include spherical calcium carbonate. From the viewpoints of improving suppression of powder scattering, ease of taking an appropriate amount, good spreadability, and moldability in a well-balanced manner, the content of spherical calcium carbonate in the cosmetic base material may be, based on the total amount of the cosmetic base material, 0.1 to 10% by mass, 1 to 7% by mass, or 3 to 5% by mass.

From the viewpoints of improving suppression of powder scattering, ease of taking an appropriate amount, transparency of finish, good spreadability, and moldability in a well-balanced manner, the total content of the component (A) and spherical calcium carbonate in the cosmetic base material may be, based on the total amount of the cosmetic base material, 1 to 50% by mass, 5 to 47% by mass, or 20 to 45% by mass.

The cosmetic base material of the present embodiment may include powder components other than the component (A) and the component (B) (sometimes referred to as other powder components). The other powder components are not particularly limited as long as they are usually used in cosmetics, and examples thereof include extender powders, white pigments, and color pigments. The shape of the other powder components is not particularly limited as long as it is not spherical, and the other powder components may have, for example, a shape such as a plate-like shape or a needle-like shape, a fumed shape, a fine particle diameter, a pigment-grade particle diameter, and a particle structure such as a porous structure or a non-porous structure.

Specific examples thereof include extender pigments such as mica, synthetic mica, sericite, talc, kaolin, boron nitride, silicon carbide, barium sulfate, aluminum oxide, silica, magnesium oxide, zirconium oxide, magnesium carbonate, calcium carbonate, chromium oxide, and magnesium aluminum hydroxide; white pigments such as titanium oxide and zinc oxide; organic powders such as polyethylene powder, polystyrene powder, organopolysiloxane elastomer powder, polymethylsilsesquioxane powder, urethane powder, wool powder, silk powder, cellulose powder, and N-acyl lysine powder; composite powders such as titanium mica coated with fine titanium oxide, nylon coated with fine titanium oxide, titanium mica coated with barium sulfate, silica containing titanium oxide, silica containing zinc oxide, and alumina-coated mica.

Examples of color pigments include inorganic color pigments such as red iron oxide, yellow iron oxide, black iron oxide, cobalt oxide, chromium oxide, ultramarine, Prussian blue, titanium oxide, and zinc oxide; organic color pigments such as aluminum lakes of Red No. 228, Red No. 226, Blue No. 404, Red No. 202, and Yellow No. 4; pearl pigments such as titanium mica, fish scale foil, bismuth oxychloride, and aluminum flakes; and natural dyes such as carmine and safflower.

From the viewpoint of moldability, The cosmetic base material may include, as the other powder components, at least one of calcium carbonate and magnesium carbonate having a non-spherical shape. From the viewpoint of moldability, the total content of calcium carbonate and magnesium carbonate having a non-spherical shape in the cosmetic base material may be, based on the total amount of the cosmetic base material, 1 to 10% by mass, 2 to 8% by mass, or 3 to 6% by mass.

When the cosmetic base material includes spherical calcium carbonate or magnesium carbonate and calcium carbonate or magnesium carbonate having a non-spherical shape, from the viewpoint of moldability, the total content of calcium carbonate and magnesium carbonate in the cosmetic base material may be, based on the total amount of the cosmetic base material, 1 to 10% by mass, 2 to 8% by mass, or 3 to 6% by mass.

The cosmetic base material may include, as the other powder components, a powder component having an ultraviolet scattering effect (also referred to as an ultraviolet scattering agent), or may not include an ultraviolet scattering agent. Examples of the ultraviolet scattering agent include fine titanium oxide and zinc oxide. The content of ultraviolet scattering agent in the cosmetic base material may be, based on the total amount of the cosmetic base material, 1 to 40% by mass, 3 to 30% by mass, or 5 to 25% by mass.

The other powder components may be surface-treated. The surface treatment can be employed without particular limitation as long as it is used in cosmetics, and examples thereof include surface treatment with a metal soap, a higher fatty acid, an oil or fat, a wax, a silicone compound, a fluorine compound, a surfactant, or a dextrin fatty acid ester.

The content of powder component in the cosmetic base material may be, from the viewpoint of good spreadability, based on the total amount of the cosmetic base material, 60 to 96% by mass, 65 to 93% by mass, or 70 to 90% by mass.

### <Thickener>

In the present embodiment, the cosmetic base material further includes (C) a water-soluble thickener (sometimes referred to as a component (C)).

The component (C) is an additive that increases the viscosity of a liquid by being dissolved in water, and can be used without particular limitation as long as it is used in cosmetics.

From the viewpoints of suppression of powder scattering, adhesion to the skin, transparency of finish, and impact resistance, the component (C) may include one or more thickeners selected from the group consisting of an acrylic acid copolymer and a clay mineral.

When the cosmetic base material includes an acrylic acid copolymer as the component (C), suppression of powder scattering and improvement in drop strength and adhesion to the skin can be achieved at a higher level. The reason for this is thought to be that the acrylic acid copolymer improves dispersibility of the powder in the slurry and filling properties of the slurry, and functions as a binder in a molded body (solid powder cosmetic) after the dispersion medium is removed. Examples of the acrylic acid copolymer include (hydroxyethyl acrylate/sodium acryloyldimethyltaurate) copolymer, (sodium acrylate/sodium acryloyldimethyltaurine) copolymer, and sodium acrylate graft starch.

When the cosmetic base material includes a clay mineral as the component (C), suppression of powder scattering and improvement in drop strength can also be achieved at a higher level. The reason for this is thought to be that the clay mineral improves dispersibility of the powder in the slurry and filling properties of the slurry, and functions as a binder in a molded body (solid powder cosmetic) after the dispersion medium is removed. Examples of the clay mineral include bentonite, magnesium aluminum silicate, and hectorite.

From the viewpoints of improving suppression of powder scattering, ease of taking an appropriate amount, adhesion to the skin, transparency of finish, and impact resistance in a well-balanced manner, the content of the component (C) in the cosmetic base material may be, based on the total amount of the cosmetic base material, 0.01 to 10% by mass or 0.05 to 5% by mass.

When the cosmetic base material includes an acrylic acid copolymer as the component (C), the content of the acrylic acid copolymer, from the viewpoints of improving suppression of powder scattering, ease of taking an appropriate amount, adhesion to the skin, transparency of finish, and impact resistance in a well-balanced manner, may be, based on the total amount of the cosmetic base material, 0.01 to 0.5% by mass, 0.05 to 0.4% by mass, or 0.10 to 0.25% by mass.

When the cosmetic base material includes a clay mineral as the component (C), the content of the clay mineral, from the viewpoints of improving suppression of powder scattering, ease of taking an appropriate amount, adhesion to the skin, transparency of finish, and impact resistance in a well-balanced manner, may be, based on the total amount of the cosmetic base material, 1 to 10% by mass, 2 to 8% by mass, or 3 to 6% by mass.

### <Oily Component>

In the present embodiment, the oily component includes (D) a water-holding oil agent (sometimes referred to as a component (D)). The water-holding oil agent means an oil agent having a water-holding capacity of an amount equal to or more than its own weight (100% or more).

The component (D) may be an oil agent having a water-holding capacity of 100% or more at room temperature (25° C.). In addition, the component (D) may be a liquid oil or a semi-solid oil, and may be a semi-solid oil.

In the present specification, the term “liquid oil” refers to an oil in a liquid state at 25° C., and the term “semi-solid oil” refers to an oil in a paste state at 25° C.

Examples of the component (D) include liquid oils such as di(isostearyl/phytosteryl) dimer dilinoleate and (cholesterol/lanosterol) fatty acid (C10-30) esters; and semi-solid oils such as (phytosteryl/isostearyl/cetyl/stearyl/behenyl) dimer dilinoleate, hydrogenated castor oil isostearate, bis-behenyl/isostearyl/phytosteryl dimer dilinoleyl dimer dilinoleate, dipentaerythrityl hexa (hydroxystearate/stearate/rosinate), and dipentaerythrityl tetra(hydroxystearate/isostearate). Among these, from the viewpoint of improving usability, semi-solid oils of phytosterol derivatives such as (phytosteryl/isostearyl/cetyl/stearyl/behenyl) dimer dilinoleate and bis-behenyl/isostearyl/phytosteryl dimer dilinoleyl dimer dilinoleate may be used.

The content of the component (D) in the cosmetic base material may be, from the viewpoint of adhesion to the skin, based on the total amount of the cosmetic base material, 1 to 10% by mass, 2 to 8% by mass, or 3 to 6% by mass.

The content of the component (D) in the cosmetic base material may be, from the viewpoints of ease of taking an appropriate amount and moldability, based on the total amount of the oily component, 50% by mass or less, 40% by mass or less, 35% by mass or less, or 33% by mass or less. From the viewpoints of adhesion to the skin and impact resistance, the above content may be 1% by mass or more, 10% by mass or more, or 25% by mass or more.

The cosmetic base material of the present embodiment may include oily components other than the component (D) (sometimes referred to as other oily components). The other oily components are not particularly limited as long as they are usually used in cosmetics, and solid oils, semi-solid oils, and liquid oils can be used.

Examples of liquid oils (hereinafter also referred to as other liquid oils) include: ester oils such as cetyl ethylhexanoate, ethylhexyl palmitate, triethylhexanoin, acid isotridecyl isononanoate, isostearyl isostearate, neopentyl glycol diethylhexanoate, polyglyceryl-2 isostearate, (caprylic/capric) triglyceride, neopentyl glycol dicaprate, propanediol diisostearate, trimethylolpropane triethylhexanoate, octyldodecyl myristate, octyldodecyl stearoyloxy stearate, diisostearyl malate, polyglyceryl triisostearate, dipentaerythrityl penta-isostearate, and trimethylolpropane triisostearate; hydrocarbon oils such as olefin oligomers, liquid paraffin, squalane, and hydrogenated polyisobutene; silicone oils such as dimethicone; vegetable oils such as sunflower seed oil, jojoba seed oil, olive oil, and castor oil; and higher alcohols such as isostearyl alcohol, octyldodecanol, and oleyl alcohol.

From the viewpoints of improving suppression of powder scattering, ease of taking an appropriate amount, and good spreadability in a well-balanced manner, the content of other liquid oils in the cosmetic base material may be, based on the total amount of the oily component, 50 to 95% by mass, 60 to 80% by mass, or 65 to 75% by mass.

The other liquid oils may be a low-viscosity liquid oil having a viscosity of less than 50 mPa·s at 25° C., a medium-viscosity liquid oil having a viscosity of 50 mPa·s to 1000 mPa·s at 25° C., a high-viscosity liquid oil having a viscosity exceeding 1000 mPa·s at 25° C., or a combination thereof. When the cosmetic base material includes other liquid oils, the other liquid oils may include a low-viscosity liquid oil, or a low-viscosity liquid oil and a medium-viscosity liquid oil, from the viewpoints of good spreadability and a natural finish.

In the present specification, the viscosity of a liquid oil at 25° C. means a value measured for a sample at 25° C. using a Brookfield-type viscometer (BM type) under the following conditions. The measurement time is 1 minute.
5 to 50 mPa·s: BM type, BL adapter, rotation speed 12 rpm
50 to 500 mPa·s: BM type, rotor No. 1, rotation speed 12 rpm
250 to 2500 mPa·s: BM type, rotor No. 2, rotation speed 12 rpm
1000 to 10000 mPa·s: BM type, rotor No. 3, rotation speed 12 rpm
5000 to 50000 mPa·s: BM type, rotor No. 4, rotation speed 12 rpm

From the viewpoints of improving ease of taking an appropriate amount, adhesion to the skin, and good spreadability in a well-balanced manner, the content of the above low-viscosity liquid oil may be, based on the total amount of the oily component, 20 to 95% by mass, 35 to 80% by mass, or 45 to 75% by mass.

From the viewpoints of improving ease of taking an appropriate amount, adhesion to the skin, and good spreadability in a well-balanced manner, the content of the above low-viscosity liquid oil may be 50 to 300 parts by mass, 70 to 250 parts by mass, or 90 to 200 parts by mass relative to 100 parts by mass of the component (D).

From the viewpoints of improving ease of taking an appropriate amount, adhesion to the skin, and good spreadability in a well-balanced manner, the content of the above medium-viscosity liquid oil may be, based on the total amount of the oily component, 0.1 to 80% by mass, 5 to 70% by mass, or 10 to 60% by mass.

From the viewpoints of improving ease of taking an appropriate amount, adhesion to the skin, and good spreadability in a well-balanced manner, the content of the above medium-viscosity liquid oil may be 0.1 to 200 parts by mass, 1 to 150 parts by mass, or 10 to 100 parts by mass relative to 100 parts by mass of the component (D). The cosmetic base material of the present embodiment may not include a medium-viscosity liquid oil as the other oily component.

From the viewpoints of improving ease of taking an appropriate amount, adhesion to the skin, and good spreadability in a well-balanced manner, the content of the above high-viscosity liquid oil may be, based on the total amount of the oily component, 0.1 to 80% by mass, 0.5 to 70% by mass, or 1 to 60% by mass. The cosmetic base material of the present embodiment may not include a high-viscosity liquid oil as the other oily component.

From the viewpoints of improving ease of taking an appropriate amount, adhesion to the skin, and good spreadability in a well-balanced manner, the contents of the above low-viscosity liquid oil and the above medium-viscosity liquid oil may respectively be, based on the total amount of the other oily components, 50 to 100% by mass and 0 to 50% by mass, 60 to 80% by mass and 20 to 40% by mass, or 65 to 75% by mass and 25 to 35% by mass.

The other liquid oils may be a non-polar oil, a polar oil, or a combination thereof. When the cosmetic base material includes other liquid oils, the other liquid oils may include a non-polar oil from the viewpoints of good spreadability and a natural finish. Examples of the non-polar oil include ester oils such as cetyl ethylhexanoate, ethylhexyl palmitate, triethylhexanoin, isotridecyl isononanoate, isostearyl isostearate, neopentyl glycol diethylhexanoate, (caprylic/capric) triglyceride, neopentyl glycol dicaprate, trimethylolpropane triethylhexanoate, octyldodecyl myristate, and trimethylolpropane triisostearate; hydrocarbon oils such as olefin oligomers, liquid paraffin, squalane, and hydrogenated polyisobutene; and silicone oils such as dimethicone.

From the viewpoints of improving good spreadability and a natural finish in a well-balanced manner, the content of the above non-polar oil may be, based on the total amount of the oily component, 50 to 95% by mass, 60 to 80% by mass, or 65 to 75% by mass.

Examples of semi-solid oils include petrolatum, (caprylic/capric/myristic/stearic) triglyceride, sucrose rosin pentaerythritol ester, and hexa (oleate/palmitate/stearate), cholesterol fatty acid esters.

From the viewpoints of improving adhesion to the skin and maintaining drop strength, the content of the above semi-solid oil may be, based on the total amount of the oily component, 0.1 to 8.0% by mass, 0.5 to 7.0% by mass, or 1.0 to 5.0% by mass. The cosmetic base material of the present embodiment may not include the above semi-solid oil as the other oily component.

Examples of solid oils include hydrocarbons such as paraffin wax, microcrystalline wax, and polyethylene; plant-derived oils and fats such as hardened castor oil (hydrogenated castor oil), hydrogenated jojoba oil, carnauba wax, candelilla wax, candelilla wax hydrocarbon, sunflower seed wax, and rice wax; ester oils such as glyceryl tribehenate; higher alcohols such as stearyl alcohol and behenyl alcohol; silicones such as acrylic-modified silicone; and sugar fatty acid esters such as dextrin palmitate and inulin stearate.

From the viewpoint of maintaining drop strength, the content of the above solid oil may be, based on the total amount of the oily component, 0.1 to 5% by mass, 0.2 to 3% by mass, or 0.5 to 1% by mass. The cosmetic base material of the present embodiment may not contain a solid oil as the other oily component.

From the viewpoints of improving ease of taking an appropriate amount, good spreadability, and adhesion to the skin in a well-balanced manner, the content of the oily component in the cosmetic base material may be, based on the total amount of the cosmetic base material, 4 to 40% by mass, 7 to 35% by mass, or 10 to 30% by mass.

From the viewpoints of improving suppression of powder scattering, ease of taking an appropriate amount, and adhesion to the skin in a well-balanced manner, the content of semi-solid oil in the cosmetic base material, that is, the total content of the component (D) that is semi-solid and semi-solid oils other than the component (D), may be, based on the total amount of the cosmetic base material, 1 to 6% by mass, 2 to 5.5% by mass, 3 to 5% by mass, or 4 to 4.5% by mass.

### <Other Components>

The cosmetic base material of the present embodiment may further include, as other components in addition to the above-described components, additives usually used in cosmetics. Examples of additives include polyhydric alcohols, surfactants, antioxidants such as tocopherol, preservatives such as ethylparaben, methylparaben, and chlorphenesin, perfume, and beauty ingredients. The surfactants are may be nonionic surfactants having a low HLB (for example, an HLB of 3 to 6), and may be fatty acid sorbitan-based nonionic surfactants. Examples of fatty acid sorbitan-based nonionic surfactants include sorbitan sesquiisostearate.

Examples of polyhydric alcohols include dipropylene glycol, 1,3-butylene glycol, isopentyldiol, 1,2-pentanediol, 1,2-hexanediol, polyethylene glycol, glycerin, diglycerin, sorbitol, maltitol, and raffinose.

From the viewpoint of storage stability capable of suppressing a decrease in hardness even in a high-temperature and high-humidity environment, the content of the polyhydric alcohol in the cosmetic base material may be, based on the total amount of the cosmetic base material, less than 0.1% by mass, and may be 0.05% by mass or less, or 0.01% by mass or less. The cosmetic base material of the present embodiment may not include a polyhydric alcohol.

Next, the dispersion medium contained in the slurry will be described.

### <Dispersion Medium>

In the present embodiment, the dispersion medium contains water. The dispersion medium may contain only water, or may contain water and a volatile solvent other than water.

Examples of the volatile solvent other than water include light liquid isoparaffin, ethyl alcohol, acetone, and isopropyl alcohol.

The content of water in the dispersion medium may be, based on the total amount of the dispersion medium, 50 to 100% by mass, 80 to 100% by mass, or 100% by mass.

Next, a method for preparing the slurry will be described.

### <Preparation of Slurry>

In Step A, a slurry containing a cosmetic base material including the above-described components and a dispersion medium containing water is prepared. The slurry may be prepared by mixing a mixture of the components of the cosmetic base material with the dispersion medium, by adding the components of the cosmetic base material to the dispersion medium in a predetermined order, or by preparing two or more mixtures of some of the components of the cosmetic base material and the dispersion medium and mixing these.

Examples of mixing devices include a Disper, a Homomixer, a Supermixer, a Henschel mixer, and an atomizer.

In Step A, heating may be performed, and defoaming of the slurry may be performed, if necessary.

When mixing the mixture of the components of the cosmetic base material with the dispersion medium, Step A may include, for example, a step of obtaining a first mixture in which the components of the cosmetic base material other than the oily component (powder components, a thickener, and other components) are mixed in a predetermined ratio, and a step of obtaining a second mixture in which oily components are mixed in a predetermined ratio.

The step of obtaining the first mixture and second mixture may be performed using, for example, a Disper or a Homomixer. In the step of obtaining the second mixture, the oily components may be mixed while being heated at 60° C. to 80° C. In the step of obtaining the second mixture, the oily components may be mixed while heating at 60-80° C. or at 60-70° C.

The step of mixing the first mixture and the second mixture may be performed using, for example, a Supermixer or a Henschel mixer, and pulverization may be performed using an atomizer or the like, if necessary.

The blending ratio of the cosmetic base material and the dispersion medium may be, by mass ratio, cosmetic base material: dispersion medium=100:100 to 100:10, 100:100 to 100:40, or 100:100 to 100:80.

### (Step B)

Next, Step B will be described.

Step B may include a step of filling the slurry into a container and a step of removing the dispersion medium from the slurry filled in the container to perform molding.

Examples of the predetermined container include middle pans such as a metal pan and a resin pan.

The step of removing the dispersion medium from the slurry filled in the container to perform molding may be performed, for example, by performing molding and then removing the solvent, by removing the solvent and then performing molding, or by simultaneously removing the solvent and performing molding. Examples of the molding method include compression molding using, for example, a suction compression molding machine. Examples of the solvent removal method include drying using a hot-air dryer, a freeze dryer, or the like.

Through Step A and Step B described above, a solid powder cosmetic is obtained.

The solid powder cosmetic obtained by the method of the present embodiment can be used as a makeup cosmetic such as face powder, foundation, eye shadow, blush, powder eyebrow, or nose shadow.

The solid powder cosmetic obtained by the method of the present embodiment can be applied to the skin or eyebrows using an applicator. Examples of the applicator include those in the form of a pen, a tip, a puff, or a brush.

### [Solid Powder Cosmetic]

The solid powder cosmetic of the present embodiment is a solid powder cosmetic including powder components and oily components, in which the powder components include a plate-like powder including (A) alumina and (B) spherical powder, and further including (C) a water-soluble thickener, in which the oily components include (D) a water-holding oil agent.

The solid powder cosmetic of the present embodiment can be produced by the method for producing a solid powder cosmetic of the present embodiment described above, and can have excellent usability (reduced powder scattering and ease of taking an appropriate amount), excellent feeling of finish (adhesion to the skin and a transparent finish), sufficient impact resistance, and sufficient storage stability.

The components constituting the solid powder cosmetic of the present embodiment and their contents may be the same as those of the cosmetic base material in the method for producing the solid powder cosmetic of the present embodiment described above.

The solid powder cosmetic of the present embodiment may have a hardness, at 25° C. measured under the following conditions using a load tester (Rheometer, manufactured by RHEOTECH), of 0.1 to 10 N, 0.5 to 5 N, or 1 to 3 N.

Measurement device: Rheometer (manufactured by RHEOTECH)

### [Measurement Conditions]

Adapter: φ 1.0 mm cylinder
Penetration depth: 1.0 mm
Penetration speed: 2 cm/min

The solid powder cosmetic of the present embodiment can be used as a makeup cosmetic such as face powder, foundation, eye shadow, blush, powder eyebrow, or nose shadow.

The solid powder cosmetic of the present embodiment can be applied to the skin or eyebrows using an applicator because it has excellent usability (reduced powder scattering and ease of taking an appropriate amount). Examples of the applicator include those in the form of a pen, a tip, a puff, or a brush.

### Examples

Hereinafter, the present invention will be described in more detail with reference to examples. However, the technical scope of the present invention is not limited by these examples. Unless otherwise specified, the numerical values in the tables represent the content (parts by mass) when the total amount of the cosmetic base material is taken as 100 parts by mass.

Prior to the examples, evaluation methods adopted in each example will be described.

### (1) Sensory Evaluation

Ten expert cosmetic evaluation panelists were asked to apply samples of solid powder cosmetics of examples and comparative examples to their skin using brushes as an applicator. Each panelist conducted a 5-point scale evaluation according to the evaluation criteria below with respect to absence of powder scattering, ease of adjusting the amount adhering to the applicator, adhesion to the skin, transparency of finish, and good spreadability. The average score was then determined according to the criteria below.

### [Score: Evaluation Criteria]

5 Points: Very good
4 Points: Good
3 Points: Average
2 Points: Slightly poor
1 Point: Poor

### [Determination Criteria (Average Score)]

A: 4 or more
B: 3 or more and less than 4
C: 2 or more and less than 3
D: Less than 2

### (2) Moldability

For molded articles obtained by compression molding and drying, the presence or absence of cracks, peeling, fissures, or the like occurring on the surface of each sample of the solid powder cosmetic was visually observed. The occurrence level was evaluated in stages according to the evaluation criteria below, and each sample was given a score. The average score at n=5 was then determined according to the criteria below.

### [Score: Evaluation Criteria]

4 Points: No change
3 Points: Slight occurrence of cracks, peeling, fissures, or the like was observed
2 Points: Occurrence of cracks, peeling, fissures, or the like was observed
1 Point: Very frequent occurrence of cracks, peeling, fissures, or the like was observed

### [Determination Criteria (Average Score)]

A: 3.5 or more
B: 3.0 or more and less than 3.5
C: 2.0 or more and less than 3.0
D: Less than 2.0

### (3) Impact Resistance

Each sample of the solid powder cosmetic was dropped five times from a height of 50 cm onto a P-tile with the contents facing upward, and the state after dropping was observed. The occurrence of cracks, chipping, collapse, or the like was evaluated on a 4-point scale according to the evaluation criteria below, and each sample was given a score. The average score at n=5 was then determined according to the criteria below.

### [Score: Evaluation Criteria]

4 Points: No change
3 Points: Cracks were observed
2 Points: Chipping was observed
1 Point: Collapse of more than half of the sample was observed

### [Determination Criteria (Average Score)]

A: 3.5 or more
B: 3.0 or more and less than 3.5
C: 2.0 or more and less than 3.0
D: Less than 2.0

### (4) Storage Stability

The samples of the solid powder cosmetics were stored for four weeks in a constant-temperature chamber set at a temperature of 40° C. and a humidity of 70%. The hardness of the solid powder cosmetics before and after storage was measured under the measurement conditions below using a load tester (Rheometer, manufactured by RHEOTECH), and the average value at n=5 was calculated. Based on the rate of decrease in hardness after storage relative to an initial value, with the hardness before storage as the initial value ([(initial value)−(hardness after storage)]×100/(initial value)), the storage stability was determined on a 3-point scale according to the determination criteria below.

### [Measurement Conditions]

Adapter: φ 1.0 mm cylinder
Penetration depth: 1.0 mm
Penetration speed: 2 cm/min
Measurement temperature: 25° C.

### [Determination Criteria]

1: Rate of decrease in hardness is less than 10%
2: Rate of decrease in hardness is 10% or more and less than 15%
3: Rate of decrease in hardness is 15% or more

### Examples 1 to 32 and Comparative Examples 1 to 6

Solid powder cosmetics (face powders) having the compositions shown in Tables 1 to 5 were prepared by the following production method and evaluated as described above. The results are collectively shown in Tables 1 to 5.

### <Production Method>

Oily components were heated and mixed at 60° C. to 80° C. to obtain a mixture I. Materials (powder components, thickeners, and other components) of the cosmetic base material other than the oily components were uniformly dispersed using a Henschel mixer to obtain a mixture II. The mixture I was added to the mixture II, and the resulting mixture was uniformly dispersed using a Henschel mixer to obtain a cosmetic base material. To 100 parts by mass of this cosmetic base material, water as a solvent was added in an amount such that the parts by mass were as shown in Tables 1 to 5, and kneaded to form a slurry. The slurry was then filled into a middle pan (metal pan), compression-molded, and dried to remove the water, thereby producing a face powder.

TABLE 1

| | | | | Examples | | | | |
|---|---|---|---|---|---|---|---|---|
| | | | | 1 | 2 | 3 | 4 | 5 |
| Cosmetic base | Powder | (A) | (A1) Alumina | 15 | 15 | 15 | 15 | 15 |
| materials | components | | single crystal | | | | | |
| | | | (A2) Alumina- | — | — | — | — | — |
| | | | based pearlescent | | | | | |
| | | | agent | | | | | |
| | | (B) | Spherical silica 1 | 8 | 8 | 8 | 8 | 8 |
| | | | Spherical silica 2 | 8 | 8 | 8 | 8 | 8 |
| | | Others | Calcium carbonate | 6 | 6 | 6 | 6 | 6 |
| | | | Synthetic mica 1 | Balance | Balance | Balance | Balance | Balance |
| | | | Synthetic mica 2 | 20 | 20 | 20 | 20 | 20 |
| | | | Boron nitride | — | — | — | — | — |
| | | | Silicone-treated | 0.4 | 0.4 | 0.4 | 0.4 | 0.4 |
| | | | colorant | | | | | |
| | Thickener | (C) | Acrylic polymer 1 | 0.05 | 0.1 | 0.15 | 0.25 | 0.3 |
| | | | Acrylic polymer 2 | — | — | — | — | — |
| | | | Bentonite | — | — | — | — | — |
| | | | Al—Mg silicate | — | — | — | — | — |
| | Oily | (D) | Water-holding oil | 4 | 4 | 4 | 4 | 4 |
| | components | | agent | | | | | |
| | | Others | Silicone oil 1 | 3 | 3 | 3 | 3 | 3 |
| | | | Ester oil 1 | 7 | 7 | 7 | 7 | 7 |
| | Others | Surfactant | | 0.1 | 0.1 | 0.1 | 0.1 | 0.1 |
| | | Antioxidant | | 0.02 | 0.02 | 0.02 | 0.02 | 0.02 |
| | | Preservative | | 0.2 | 0.2 | 0.2 | 0.2 | 0.2 |
| | Content (parts by mass) | | | 100 | 100 | 100 | 100 | 100 |
| Dispersion | Water (parts by mass) | | | 80 | 80 | 80 | 80 | 80 |
| medium | | | | | | | | |
| Evaluation | Absence of powder scattering, ease | | | B | B | A | A | C |
| | of taking appropriate amount | | | | | | | |
| | Adhesion to skin | | | B | A | A | A | B |
| | Transparency of finish | | | B | A | A | A | B |
| | Good spreadability | | | A | A | A | A | A |
| | Moldability | | | A | A | A | A | B |
| | Impact resistance | | | B | A | A | A | A |
| | Storage stability | | | 1 | 1 | 1 | 1 | 1 |
| | | | | Examples | | | | |
| | | | | 6 | 7 | 8 | 9 | 10 |
| Cosmetic base | Powder | (A) | (A1) Alumina | 15 | 15 | 15 | 15 | 15 |
| materials | components | | single crystal | | | | | |
| | | | (A2) Alumina- | — | — | — | — | 5 |
| | | | based pearlescent | | | | | |
| | | | agent | | | | | |
| | | (B) | Spherical silica 1 | 8 | 8 | 8 | 8 | 8 |
| | | | Spherical silica 2 | 8 | 8 | 8 | 8 | 12 |
| | | Others | Calcium carbonate | 6 | 6 | 6 | 4 | 5 |
| | | | Synthetic mica 1 | Balance | Balance | Balance | Balance | Balance |
| | | | Synthetic mica 2 | 20 | 20 | 20 | 15 | |
| | | | Boron nitride | — | — | — | 4 | — |
| | | | Silicone-treated | 0.4 | 0.4 | 0.4 | 1 | 0.3 |
| | | | colorant | | | | | |
| | Thickener | (C) | Acrylic polymer 1 | — | — | — | — | 0.2 |
| | | | Acrylic polymer 2 | 0.15 | — | — | 0.15 | — |
| | | | Bentonite | — | 5 | — | — | — |
| | | | Al—Mg silicate | — | — | 5 | — | — |
| | Oily | (D) | Water-holding oil | 4 | 4 | 4 | 4 | 4.5 |
| | components | | agent | | | | | |
| | | Others | Silicone oil 1 | 3 | 3 | 3 | 3 | 3 |
| | | | Ester oil 1 | 7 | 7 | 7 | 7 | 7 |
| | Others | Surfactant | | 0.1 | 0.1 | 0.1 | 0.1 | 0.1 |
| | | Antioxidant | | 0.02 | 0.02 | 0.02 | 0.02 | 0.02 |
| | | Preservative | | 0.2 | 0.2 | 0.2 | 0.2 | 0.2 |
| | Content (parts by mass) | | | 100 | 100 | 100 | 100 | 100 |
| Dispersion | Water (parts by mass) | | | 80 | 80 | 80 | 80 | 80 |
| medium | | | | | | | | |

| | | | | | | |
|---|---|---|---|---|---|---|
| Evaluation | Absence of powder scattering, ease of | A | A | A | A | A |
| | taking appropriate amount | | | | | |
| | Adhesion to skin | A | C | C | A | A |
| | Transparency of finish | A | C | C | A | A |
| | Good spreadability | A | A | A | A | A |
| | Moldability | A | C | C | A | A |
| | Impact resistance | A | A | A | A | A |
| | Storage stability | 1 | 1 | 1 | 1 | 1 |

TABLE 2

| | | | | Examples | | | | |
|---|---|---|---|---|---|---|---|---|
| | | | | 11 | 12 | 13 | 14 | 15 |
| Cosmetic base | Powder | (A) | (A1) Alumina single crystal | 15 | 15 | 15 | 15 | 15 |
| materials | components | | (A2) Alumina-based | 5 | 5 | 5 | 5 | 5 |
| | | | pearlescent agent | | | | | |
| | | (B) | Spherical silica 1 | 8 | 8 | 8 | 8 | 8 |
| | | | Spherical silica 2 | 12 | 12 | 12 | 12 | 12 |
| | | Others | Calcium carbonate | 5 | 5 | 5 | 5 | 5 |
| | | | Synthetic mica 1 | Balance | Balance | Balance | Balance | Balance |
| | | | Synthetic mica 2 | | | | | |
| | | | Silicone-treated colorant | 0.3 | 0.3 | 0.3 | 0.3 | 0.3 |
| | | | Pearlescent agent | — | — | — | — | — |
| | Thickener | (C) | Acrylic polymer 1 | 0.2 | 0.2 | 0.2 | 0.2 | 0.2 |
| | | | Acrylic polymer 2 | — | — | — | — | — |
| | Oily components | (D) | Water-holding oil agent 1 | — | — | — | 4.5 | 4.5 |
| | | | Water-holding oil agent 2 | 4.5 | — | — | — | — |
| | | | Water-holding oil agent 3 | — | 4.5 | — | — | — |
| | | | Water-holding oil agent 4 | — | — | 4.5 | — | — |
| | | | Water-holding oil agent 5 | — | — | — | — | — |
| | | Others | Silicone oil 1 | 3 | 3 | 3 | 3 | 3 |
| | | | Ester oil 3 | 7 | 7 | 7 | — | — |
| | | | Ester oil 2 | — | — | — | — | 7 |
| | | | Ester oil 3 | — | — | — | 7 | — |
| | Others | Surfactant | | 0.1 | 0.1 | 0.1 | 0.1 | 0.1 |
| | | Antioxidant | | 0.02 | 0.02 | 0.02 | 0.02 | 0.02 |
| | | Preservative | | 0.2 | 0.2 | 0.2 | 0.2 | 0.2 |
| | Content (parts by mass) | | | 100 | 100 | 100 | 100 | 100 |
| Dispersion medium | Water (parts by mass) | | | 80 | 80 | 80 | 80 | 80 |
| Evaluation | Absence of powder scattering, ease | | | A | A | A | A | B |
| | of taking appropriate amount | | | | | | | |
| | Adhesion to skin | | | A | A | B | A | A |
| | Transparency of finish | | | A | A | A | A | A |
| | Good spreadability | | | A | A | A | A | A |
| | Moldability | | | A | A | B | C | C |
| | Impact resistance | | | A | A | A | C | C |
| | Storage stability | | | 1 | 1 | 1 | 1 | 1 |
| | | | | Examples | | | | |
| | | | | 16 | 17 | 18 | 19 | 20 |
| Cosmetic base | Powder | (A) | (A1) Alumina single crystal | 15 | 15 | 15 | 5 | 18 |
| materials | components | | (A2) Alumina-based | 5 | 5 | 5 | 36 | — |
| | | | pearlescent agent | | | | | |
| | | (B) | Spherical silica 1 | 8 | 8 | 8 | 4 | 16 |
| | | | Spherical silica 2 | 12 | 12 | 12 | — | — |
| | | Others | Calcium carbonate | 5 | 5 | 5 | 3 | 5 |
| | | | Synthetic mica 1 | Balance | Balance | Balance | Balance | Balance |
| | | | Synthetic mica 2 | | | | | 20 |
| | | | Silicone-treated colorant | 0.3 | 0.3 | 0.3 | 0.3 | 0.4 |
| | | | Pearlescent agent | — | — | — | 8 | — |
| | Thickener | (C) | Acrylic polymer 1 | 0.2 | 0.2 | 0.2 | — | — |
| | | | Acrylic polymer 2 | — | — | — | 0.22 | 0.2 |
| | Oily components | (D) | Water-holding oil agent 1 | — | — | — | 5 | 4 |
| | | | Water-holding oil agent 2 | 4.5 | 4.5 | — | — | — |
| | | | Water-holding oil agent 3 | — | — | 3 | — | — |
| | | | Water-holding oil agent 4 | — | — | — | — | — |
| | | | Water-holding oil agent 5 | — | — | 3 | — | — |
| | | Others | Silicone oil 1 | 3 | 3 | — | 3 | 3 |
| | | | Ester oil 3 | — | — | — | 7 | 7 |
| | | | Ester oil 2 | — | 7 | 7 | — | — |
| | | | Ester oil 3 | 7 | — | — | — | — |

| | | | | | | |
|---|---|---|---|---|---|---|
| Others | Surfactant | 0.1 | 0.1 | 0.1 | 0.1 | 0.1 |
| | Antioxidant | 0.02 | 0.02 | 0.02 | 0.02 | 0.02 |
| | Preservative | 0.2 | 0.2 | 0.2 | 0.2 | 0.2 |

| | | | | | | |
|---|---|---|---|---|---|---|
| | Content (parts by mass) | 100 | 100 | 100 | 100 | 100 |
| Dispersion medium | Water (parts by mass) | 80 | 80 | 80 | 80 | 80 |
| Evaluation | Absence of powder scattering, ease | A | B | A | A | A |
| | of taking appropriate amount | | | | | |
| | Adhesion to skin | A | A | A | A | A |
| | Transparency of finish | A | A | A | A | A |
| | Good spreadability | A | A | A | B | A |
| | Moldability | C | C | A | A | A |
| | Impact resistance | B | A | A | A | A |
| | Storage stability | 1 | 1 | 1 | 1 | 1 |

TABLE 3

| | | | | Examples | | | | |
|---|---|---|---|---|---|---|---|---|
| | | | | 21 | 22 | 23 | 24 | 25 |
| Cosmetic base | Powder | (A) | (A1) Alumina single crystal | 18 | 18 | 20 | 20 | 20 |
| materials | components | | (A2) Alumina-based | — | — | — | 4 | 4 |
| | | | pearlescent agent | | | | | |
| | | (B) | Spherical silica 1 | — | 8 | 10 | 8 | 8 |
| | | | Spherical silica 2 | 16 | 8 | 10 | 6 | 6 |
| | | | Spherical nylon | — | — | — | — | — |
| | | Others | Calcium carbonate | 5 | 5 | 5 | 6 | 6 |
| | | | Synthetic mica 1 | Balance | Balance | Balance | Balance | Balance |
| | | | Synthetic mica 2 | 20 | 20 | — | 12 | 12 |
| | | | Amino acid-treated mica | — | — | 21.18 | — | — |
| | | | Boron nitride | — | — | — | 6 | — |
| | | | Silicone-treated talc | — | — | — | — | — |
| | | | Silicone-treated colorant | 0.4 | 0.4 | 0.01 | 0.01 | 0.01 |
| | Thickener | (C) | Acrylic polymer 1 | — | — | — | — | — |
| | | | Acrylic polymer 2 | 0.2 | 0.2 | 0.2 | 0.18 | 0.18 |
| | Oily components | (D) | Water-holding oil agent 1 | 4 | 4 | 4 | — | — |
| | | | Water-holding oil agent 2 | — | — | — | 4.5 | 4.5 |
| | | | Water-holding oil agent 5 | — | — | — | — | — |
| | | Others | Silicone oil 1 | 3 | 3 | 2.38 | 3 | 3 |
| | | | Ester oil 1 | 7 | 7 | 6.5 | 7 | 7 |
| | | | Ester oil 2 | — | — | — | — | — |
| | | | Petrolatum | — | — | — | — | — |
| | Others | Surfactant | | 0.3 | 0.3 | 0.1 | 0.1 | 0.1 |
| | | Antioxidant | | 0.02 | 0.02 | 0.02 | 0.02 | 0.02 |
| | | Preservative | | 0.2 | 0.2 | 0.2 | 0.2 | 0.2 |
| | Content (parts by mass) | | | 100 | 100 | 100 | 100 | 100 |
| Dispersion medium | Water (parts by mass) | | | 80 | 80 | 80 | 80 | 80 |
| Evaluation | Absence of powder scattering, ease | | | B | A | A | A | A |
| | of taking appropriate amount | | | | | | | |
| | Adhesion to skin | | | B | A | A | A | A |
| | Transparency of finish | | | A | A | A | A | A |
| | Good spreadability | | | A | A | A | A | A |
| | Moldability | | | A | A | A | A | A |
| | Impact resistance | | | A | A | A | A | A |
| | Storage stability | | | 1 | 1 | 1 | 1 | 1 |
| | | | | Examples | | | | |
| | | | | 26 | 27 | 28 | 29 | 30 |
| Cosmetic base | Powder | (A) | (A1) Alumina single crystal | 20 | 20 | 25 | 20 | 15 |
| materials | components | | (A2) Alumina-based | 4 | 5 | — | 5 | 5 |
| | | | pearlescent agent | | | | | |
| | | (B) | Spherical silica 1 | — | 8 | 5 | 25 | 8 |
| | | | Spherical silica 2 | — | 12 | 10 | — | 12 |
| | | | Spherical nylon | 14 | — | — | — | — |
| | | Others | Calcium carbonate | 6 | 5 | 5 | 5 | 5 |
| | | | Synthetic mica 1 | Balance | Balance | Balance | Balance | Balance |
| | | | Synthetic mica 2 | 12 | — | — | 10 | — |
| | | | Amino acid-treated mica | — | — | — | — | — |
| | | | Boron nitride | — | — | — | — | — |
| | | | Silicone-treated talc | — | 10 | — | — | — |
| | | | Silicone-treated colorant | 0.01 | 0.25 | 0.4 | 0.3 | 0.3 |
| | Thickener | (C) | Acrylic polymer 1 | — | 0.2 | 0.2 | 0.3 | 0.2 |
| | | | Acrylic polymer 2 | 0.18 | — | — | — | — |
| | Oily components | (D) | Water-holding oil agent 1 | — | 4.5 | 4.5 | 4.5 | — |
| | | | Water-holding oil agent 2 | 4.5 | — | — | — | — |
| | | | Water-holding oil agent 5 | — | — | — | — | 3 |
| | | Others | Silicone oil 1 | 3 | 3 | 3 | 3 | — |
| | | | Ester oil 1 | 7 | 7 | 7 | 7 | — |
| | | | Ester oil 2 | — | — | — | — | 7 |
| | | | Petrolatum | — | — | — | — | 3 |

| | | | | | | |
|---|---|---|---|---|---|---|
| Others | Surfactant | 0.1 | 0.1 | 0.1 | 0.1 | 0.1 |
| | Antioxidant | 0.02 | 0.02 | 0.02 | 0.02 | 0.02 |
| | Preservative | 0.2 | 0.2 | 0.2 | 0.2 | 0.2 |

| | | | | | | |
|---|---|---|---|---|---|---|
| | Content (parts by mass) | 100 | 100 | 100 | 100 | 100 |
| Dispersion medium | Water (parts by mass) | 75 | 80 | 80 | 80 | 80 |
| Evaluation | Absence of powder scattering, ease | B | A | B | B | A |
| | of taking appropriate amount | | | | | |
| | Adhesion to skin | B | A | A | B | A |
| | Transparency of finish | A | B | A | A | A |
| | Good spreadability | A | A | A | A | A |
| | Moldability | A | A | A | A | A |
| | Impact resistance | A | A | A | A | A |
| | Storage stability | 1 | 1 | 1 | 1 | 1 |

TABLE 4

| | | | | Examples | |
|---|---|---|---|---|---|
| | | | | 31 | 32 |
| Cosmetic base materials | Powder components | (A) | (A1) Alumina single crystal | 12 | 15 |
| | | | (A2) Alumina-based | 16 | 5 |
| | | | pearlescent agent | | |
| | | (B) | Spherical silica 1 | 7 | 8 |
| | | | Spherical silica 2 | 3 | 12 |
| | | Others | Calcium carbonate | 6 | 5 |
| | | | Synthetic mica 1 | Balance | Balance |
| | | | Synthetic mica 2 | 8 | — |
| | | | Synthetic mica 3 | 2 | — |
| | | | Amino acid-treated mica | 10.1 | — |
| | | | Talc | 2 | — |
| | | | Silicone-treated Al | 5 | — |
| | | | hydroxide-coated mica | | |
| | | | Iron oxide | 0.2 | — |
| | | | Silicone-treated colorant | — | 0.3 |
| | | | Pearlescent agent | 10.4 | — |
| | | (C) | Acrylic polymer 1 | — | 0.2 |
| | | | Bentonite | 4 | — |
| | Oily components | (D) | Water-holding oil agent 3 | 4 | — |
| | | | Water-holding oil agent 5 | 2 | 4.5 |
| | | Others | Silicone oil 1 | — | 3 |
| | | | Ester oil 1 | — | 7 |
| | | | Ester oil 2 | 7 | — |

| | | | |
|---|---|---|---|
| Others | Surfactant | 0.1 | 0.1 |
| | Antioxidant | 0.02 | 0.02 |
| | Preservative | 0.2 | 0.2 |

| | | | |
|---|---|---|---|
| | Content (parts by mass) | 100 | 100 |
| Dispersion medium | Water (parts by mass) | 80 | 80 |
| Evaluation | Absence of powder scattering, | A | A |
| | ease of taking appropriate amount | | |
| | Adhesion to skin | B | C |
| | Transparency of finish | A | A |
| | Good spreadability | B | A |
| | Moldability | A | A |
| | Impact resistance | A | C |
| | Storage stability | 1 | 1 |

TABLE 5

| | | | | Comparative Examples | | | | | |
|---|---|---|---|---|---|---|---|---|---|
| | | | | 1 | 2 | 3 | 4 | 5 | 6 |
| Cosmetic base | Powder components | (A) | (A1) Alumina single crystal | 15 | 15 | 15 | 15 | — | — |
| materials | | (B) | Spherical silica 1 | 8 | 8 | 8 | — | 8 | 8 |
| | | | Spherical silica 2 | 8 | 8 | 8 | — | 8 | 8 |
| | | Others | Calcium carbonate | 6 | 6 | 6 | 6 | 4 | 4 |
| | | | Synthetic mica 1 | Balance | Balance | Balance | Balance | Balance | Balance |
| | | | Synthetic mica 2 | 20 | 20 | 20 | 20 | 20 | 20 |
| | | | Silicone-treated colorant | 0.4 | 0.4 | 0.4 | 0.4 | 0.4 | 0.4 |
| | Thickener | (C) | Acrylic polymer 1 | — | — | — | 0.2 | — | — |
| | | | Acrylic polymer 2 | — | — | — | — | 0.15 | — |
| | | Others | Zinc myristate | — | 2 | — | — | — | — |
| | | | Carnauba wax | — | | 0.5 | — | — | — |
| | Oily components | (D) | Water-holding oil agent | 4 | 4 | 4 | 4 | 4 | 4 |
| | | Others | Silicone oil 1 | 3 | 3 | 3 | 3 | 3 | 3 |
| | | | Ester oil 1 | 7 | 7 | 7 | 7 | 7 | 7 |

| | | | | | | | |
|---|---|---|---|---|---|---|---|
| Others | Surfactant | 0.1 | 0.1 | 0.1 | 0.1 | 0.1 | 0.1 |
| | Antioxidant | 0.02 | 0.02 | 0.02 | 0.02 | 0.02 | 0.02 |
| | Preservative | 0.2 | 0.2 | 0.2 | 0.2 | 0.2 | 0.2 |
| | Polyhydric alcohol | — | — | — | — | — | 3 |

| | | | | | | | |
|---|---|---|---|---|---|---|---|
| | Content (parts by mass) | 100 | 100 | 100 | 100 | 100 | 100 |
| Dispersion medium | Water (parts by mass) | 80 | 80 | 80 | 80 | 80 | 80 |
| Evaluation | Absence of powder scattering, ease | D | D | D | D | D | D |
| | of taking appropriate amount | | | | | | |
| | Adhesion to skin | C | C | C | B | C | C |
| | Transparency of finish | C | C | C | B | C | D |
| | Good spreadability | C | B | B | D | A | C |
| | Moldability | A | A | A | A | A | A |
| | Impact resistance | D | D | C | A | A | D |
| | Storage stability | 1 | 1 | 1 | 1 | 1 | 3 |

The details of each component in Tables 1 to 5 are as follows.

### [Powder Components]

### <Components (A)>

(A1) Alumina single crystal: Alumina single crystal (manufactured by Merck Performance Materials G.K., trade name “RonaFlair (registered trademark) White Sapphire,” average particle diameter: about 8 μm)

(A2) Alumina-based pearlescent agent: Titanium oxide-coated alumina pearlescent agent (manufactured by Merck KGaA, trade name “Ronastar (registered trademark) Golden Lights,” average particle diameter: 22.5 μm)

### <Components (B)>

Spherical silica 1: Cetanol-treated spherical silica (average particle diameter: 13 μm, surface treatment agent: 1% by mass)

Spherical silica 2: Spherical silica (average particle diameter: 7 μm, untreated)

Spherical nylon: Spherical nylon (average particle diameter: 5 μm, untreated)

### <Others>

Calcium carbonate: Calcium carbonate (plate-like, average particle diameter: 5 μm)

Synthetic mica 1: SA-treated synthetic phlogopite (manufactured by Miyoshi Kasei, Inc., average particle diameter: 10 μm) Synthetic mica 2: Synthetic mica (average particle diameter: 10 μm, untreated)

Synthetic mica 3: Synthetic mica (average particle diameter: 20 μm, untreated)

Amino acid-treated mica: NAI-treated mica (manufactured by Miyoshi Kasei, Inc., average particle diameter: about 20 μm, surface treatment agent: 1% by mass)

Silicone-treated Al hydroxide-coated mica: SA-treated aluminum hydroxide-coated mica (manufactured by Miyoshi Kasei, Inc., trade name “SA-Excel Mica JP-2,” surface treatment agent: 3% by mass) Boron nitride: Boron nitride (average particle diameter: 11 μm) Talc: Talc (average particle diameter: 6.5 μm)

Silicone-treated talc: SA-treated talc (manufactured by Miyoshi Kasei, Inc., average particle diameter: 6.5 μm)

Silicone-treated colorant: (manufactured by Miyoshi Kasei, Inc.)

Pearlescent agent: Mica-based pearlescent agent (average particle diameter: 26 μm)

### [Thickeners]

### <Components (C)>

Acrylic polymer 1: (Hydroxyethyl acrylate/sodium acryloyldimethyltaurate) copolymer (manufactured by SEPPIC S.A., product name “SEPINOV EMT 10”)

Acrylic polymer 2: (Sodium acrylate/sodium acryloyldimethyltaurate) copolymer (manufactured by SNF, trade name “FLOCARE PSD 30”) Al—Mg Silicate: Magnesium aluminum silicate

### [Oily Components]

### <Components (D)>

Water-holding oil agent 1: (Phytosteryl/isostearyl/cetyl/stearyl/behenyl) dimer dilinoleate (semi-solid oil) (manufactured by Nippon Fine Chemical Co., Ltd., trade name “Plandool-H”)

Water-holding oil agent 2: Hydrogenated castor oil isostearate (semi-solid oil) (manufactured by National Mimatsu Co., Ltd., trade name “Castride MIS-P”)

Water-holding oil agent 3: bis-behenyl/isostearyl/phytosteryl dimer dilinoleyl dimer dilinoleate (semi-solid oil) (manufactured by Nippon Fine Chemical Co., Ltd., trade name “Plandool-G”)

Water-holding oil agent 4: Dipentaerythrityl hexa (hydroxystearate/stearate/rosinate) (semi-solid oil) (manufactured by The Nisshin OilliO Group, Ltd., trade name “Cosmol 168ARV”)

Water-holding oil agent 5: Di(isostearyl/phytosteryl) dimer dilinoleate (liquid oil, viscosity: 2300 mPa·s) (manufactured by Nippon Fine Chemical Co., Ltd., trade name “LUSPLAN PI-DA”)

### <Others>

Silicone oil 1: Dimethicone (viscosity: 100 mPa·s)

Ester oil 1: Triethylhexanoin (viscosity: 31 mPa·s)

Ester oil 2: (Caprylic/capric) triglyceride (viscosity: 30 mPa·s)

Ester oil 3: Neopentyl glycol diethylhexanoate (viscosity: 12.5 mPa·s)

Petrolatum: Petrolatum (semi-solid oil) (manufactured by Nikko Rica

Corporation, trade name “Sun White (P-150)”)

The viscosity of the liquid oil was measured according to the following measurement method.

### (Viscosity Measurement Method)

A sample at 25° C. was measured using a Brookfield type viscometer (BM type) under the following conditions. The measurement time was set to 1 minute.
5 mPa·s to 50 mPa·s: BM type, BL adapter, rotation speed 12 rpm
50 to 500 mPa·s: BM type, rotor No. 1, rotation speed 12 rpm
250 to 2500 mPa·s: BM type, rotor No. 2, rotation speed 12 rpm
1000 to 10000 mPa·s: BM type, rotor No. 3, rotation speed 12 rpm
5000 to 50000 mPa·s: BM type, rotor No. 4, rotation speed 12 rpm

### [Other Components]

Surfactant: Sorbitan sesquiisostearate (viscosity: 3900 mPa·s) Polyhydric alcohol: Glycerin

As shown in Tables 1 to 5, the solid powder cosmetics obtained in Examples 1 to 32 had no evaluation marked “D” in all items, and it was confirmed that powder scattering when taken up with an applicator could be sufficiently suppressed, and adhesion to the skin, transparency of finish, impact resistance, and storage stability were all sufficient.

### Example 33: Foundation

| | (Blending ratio |
|---|---|
| (Components) | (% by mass)) |

| | | |
|---|---|---|
| 1. | Alumina single crystal | 20.00 |
| 2. | Spherical silica 1 | 8.00 |
| 3. | Spherical silica 2 | 4.00 |
| 4. | Silica 1 | 2.00 |
| 5. | Calcium carbonate | 6.00 |
| 6. | Synthetic mica 1 | Balance |
| 7. | Synthetic mica 2 | 1.50 |
| 8. | Silicone-treated Al hydroxide-coated mica | 5.00 |
| 9. | Composite powder | 5.00 |
| 10. | Fine titanium oxide | 8.50 |
| 11. | Zinc oxide | 5.00 |
| 12. | Silicone-treated colorant | 1.50 |
| 13. | Titanium oxide | 4.00 |
| 14. | Acrylic polymer 2 | 0.15 |
| 15. | Water-holding oil agent 1 | 4.00 |
| 16. | Silicone oil 1 | 3.00 |
| 17. | Ester oil 1 | 6.00 |
| 18. | Surfactant | 0.10 |
| 19. | Antioxidant | 0.02 |
| 20. | Preservative | 0.20 |

The details of the above components are the same as those described above, except for the components shown below.

[Powder components] Silica 1: Silicone-treated silica titanium oxide-encapsulated silica (average particle diameter: 4.5 μm) Composite powder: Titanium oxide-silica-alumina-mica composite powder (manufactured by Merck KGAA, trade name “RonaFlair (registered trademark) Softshade,” (average particle diameter: 10 μm) (a plate-like powder in which a mixture of titanium oxide, silica, and alumina is coated on the surface of mica)

Fine titanium oxide: Fine titanium oxide (average particle diameter: 0.14 μm)

Zinc oxide: Zinc oxide (average particle diameter: 1 μm)

Titanium oxide: Titanium oxide (average particle diameter: 0.25 μm)

### <Production Method>

Oily components were heated and mixed at 60° C. to 80° C. to obtain a mixture I. Materials (powder components, thickeners, additives, and other components) of the cosmetic base material other than the oily components were uniformly dispersed using a Henschel mixer to obtain a mixture II. The mixture I was added to the mixture II, and the resulting mixture was uniformly dispersed using a Henschel mixer to obtain a cosmetic base material. To 100 parts by mass of this cosmetic base material, 100 parts by mass of water as a solvent was added and kneaded to form a slurry. The slurry was then filled into a middle pan (metal pan), compression-molded, and dried to remove the water, thereby producing a foundation.

### <Evaluation>

The obtained sample of the foundation was evaluated in the same manner as described above, and it was confirmed that the sample received ratings of “A” for “absence of powder scattering and ease of taking an appropriate amount,” “B” for “adhesion to the skin,” “B” for “transparency of finish,” “B” for “good spreadability,” “A” for “moldability,” “A” for “impact resistance,” and “1” for “storage stability.”

### Example 34: Eye shadow

| | (Blending ratio |
|---|---|
| (Components) | (% by mass)) |

| | | |
|---|---|---|
| 1. | Alumina single crystal | 2.00 |
| 2. | Alumina-based pearlescent agent | 22.00 |
| 3. | Spherical silica 1 | 8.00 |
| 4. | Calcium carbonate | 4.00 |
| 5. | Synthetic mica 1 | Balance |
| 6. | Amino acid-treated mica | 16.77 |
| 7. | Iron oxide | 0.10 |
| 8. | Pearlescent agent | 28.00 |
| 9. | Bentonite | 4.50 |
| 10. | Water-holding oil agent 3 | 3.00 |
| 11. | Water-holding oil agent 5 | 3.00 |
| 12. | Ester oil 2 | 7.00 |
| 13. | Surfactant | 0.10 |
| 14. | Antioxidant | 0.02 |
| 15. | Preservative | 0.20 |

The details of the above components are the same as those already described above.

### <Production Method>

Oily components were heated and mixed at 60° C. to 80° C. to obtain a mixture I. Materials (powder components, thickeners, additives and other components) of the cosmetic base material other than the oily components were uniformly dispersed using a Henschel mixer to obtain a mixture II. The mixture I was added to the mixture II, and the resulting mixture was uniformly dispersed using a Henschel mixer to obtain a cosmetic base material. To 100 parts by mass of this cosmetic base material, 80 parts by mass of water as a solvent was added and kneaded to form a slurry. The slurry was then filled into a middle pan (metal pan), compression-molded, and dried to remove the water, thereby producing an eye shadow.

### <Evaluation>

The obtained sample of the eye shadow was evaluated in the same manner as described above, and it was confirmed that the sample received ratings of “A” for “absence of powder scattering and ease of taking an appropriate amount,” “B” for “adhesion to the skin,” “A” for “transparency of finish,” “B” for “good spreadability,” “A” for “moldability,” “A” for “impact resistance,” and “1” for “storage stability.”

What is claimed is:

1. A method for producing a solid powder cosmetic comprising:
a step of preparing a slurry containing a cosmetic base material including a powder component and an oily component, and a dispersion medium containing water; and
a step of filling the slurry into a container to mold the slurry by removing the dispersion medium,
wherein the powder component includes (A) at least one plate-like powder selected from the group consisting of (A1) a plate-like powder composed of alumina single crystal and (A2) a plate-like powder in which a surface of alumina is coated with a metal oxide, and (B) a spherical powder,
wherein the cosmetic base material further includes (C) a water-soluble thickener, and
wherein the oily component includes (D) a water-holding oil agent.

2. The method for producing a solid powder cosmetic according to claim 1, wherein the content of polyhydric alcohol in the cosmetic base material is less than 0.1% by mass based on the cosmetic base material.

3. The method for producing a solid powder cosmetic according to claim 1, wherein the content of the component (D) in the cosmetic base material is 50% by mass or less based on the total amount of the oily component.

4. The method for producing a solid powder cosmetic according to claim 1, wherein the component (C) includes at least one thickener selected from the group consisting of acrylic acid copolymer and a clay mineral.

5. The method for producing a solid powder cosmetic according to claim 1, wherein the component (C) includes at least one acrylic acid copolymer selected from the group consisting of (hydroxyethyl acrylate/sodium acryloyldimethyltaurate) copolymer, (sodium acrylate/sodium acryloyldimethyltaurine) copolymer, and sodium acrylate graft starch.

6. A method for producing a solid powder cosmetic according to claim 1, wherein the component (B) includes spherical particles surface-treated with a higher alcohol.

7. A method for producing a solid powder cosmetic according to claim 1, wherein the content of semi-solid oil in the cosmetic base material based on the total amount of the cosmetic base material is 1 to 6% by mass.

8. A method for producing a solid powder cosmetic according to claim 2,
wherein the content of the component (D) in the cosmetic base material is 50% by mass or less based on the total amount of the oily component,
wherein the component (C) includes at least one acrylic acid copolymer selected from the group consisting of (hydroxyethyl acrylate/sodium acryloyldimethyltaurate) copolymer, (sodium acrylate/sodium acryloyldimethyltaurine) copolymer, and sodium acrylate graft starch,
wherein the component (B) includes spherical particles surface-treated with a higher alcohol,
wherein the content of semi-solid oil in the cosmetic base material based on the total amount of the cosmetic base material is 1 to 6% by mass.

9. A solid powder cosmetic comprising:
a powder component; and
an oily component,
wherein the powder component includes (A) at least one plate-like powder selected from the group consisting of (A1) a plate-like powder composed of alumina single crystal and (A2) a plate-like powder in which a surface of alumina is coated with a metal oxide, and (B) a spherical powder,
wherein the solid powder cosmetic further includes (C) a water-soluble thickener, and
wherein the oily component includes (D) a water-holding oil agent.

10. The solid powder cosmetic according to claim 9, wherein the content of polyhydric alcohol in the solid powder cosmetic is less than 0.1% by mass based on the solid powder cosmetic.

11. The solid powder cosmetic according to claim 9, wherein the content of the component (D) in the solid powder cosmetic is 50% by mass or less based on the total amount of the oily component.

12. The solid powder cosmetic according to claim 9, wherein the component (C) includes at least one thickener selected from the group consisting of acrylic acid copolymer and a clay mineral.

13. The solid powder cosmetic according to claim 9, wherein the component (C) includes at least one acrylic acid copolymer selected from the group consisting of (hydroxyethyl acrylate/sodium acryloyldimethyltaurate) copolymer, (sodium acrylate/sodium acryloyldimethyltaurine) copolymer, and sodium acrylate graft starch.

14. The solid powder cosmetic according to claim 9, wherein the component (B) includes spherical particles surface-treated with a higher alcohol.

15. The solid powder cosmetic according to claim 9, wherein the component (B) includes spherical particles surface-treated with a higher alcohol.

16. The solid powder cosmetic according to claim 10,
wherein the content of the component (D) in the solid powder cosmetic is 50% by mass or less based on the total amount of the oily component,
wherein the component (C) includes at least one acrylic acid copolymer selected from the group consisting of (hydroxyethyl acrylate/sodium acryloyldimethyltaurate) copolymer, (sodium acrylate/sodium acryloyldimethyltaurine) copolymer, and sodium acrylate graft starch,
wherein the component (B) includes spherical particles surface-treated with a higher alcohol,
wherein the content of semi-solid oil in the solid powder cosmetic based on the total amount of the solid powder cosmetic is 1 to 6% by mass.
